(19) United States
(12) Patent Application Publication
(10) Pub. No.: US 2025/0032378 A1
(43) Pub. Date: Jan. 30, 2025

(54) PERSONAL CARE PRODUCT CONTAINING NATURAL OIL-BASED PETROLATUM

(71) Applicant: CARGILL, INCORPORATED, Wayzata, MN (US)

(72) Inventors: Emmanuel Paul Jos Marie EVERAERT, Poissy (FR); Smita FULZELE, Plymouth, MN (US); Todd L. KURTH, Maple Grove, MN (US); Serena Tess ROLLAND, Burnsville, MN (US); Yijun ZHOU, Maple Grove, MN (US)

(73) Assignee: CARGILL, INCORPORATED, Wayzata, MN (US)

(21) Appl. No.: 18/710,762

(22) PCT Filed: Nov. 16, 2022
(86) PCT No.: PCT/US2022/079954

§ 371 Date: May 16, 2024

Related U.S. Application Data
(60) Provisional application No. 63/367,352, filed on Jun. 30, 2022.
(60) Provisional application No. 63/264,211, filed on Nov. 17, 2021.

(51) Int. Cl. A61K 8/37; A61K 8/92; A61Q 1/00; A61Q 5/02; A61Q 5/12; A61Q 19/00; A61Q 19/10

(52) U.S. Cl. CPC A61K 8/37; A61K 8/92; A61Q 1/00; A61Q 5/02; A61Q 5/12; A61Q 19/00; A61Q 19/001; A61Q 19/10; A61K 2800/5922

(57) ABSTRACT

The disclosure relates to a personal care formulation comprising a natural oil-based petrolatum compositions and a method of making the same. The natural oil-based petrolatum comprises a triglyceride component wherein: the triglyceride component contains a plurality hydroxyl containing fatty acid chains and wherein a plurality of the hydroxyl containing fatty acid chains are esterified with a C8-C22 branched or straight chain fatty acid and wherein the triglyceride component: a) contains less than 10% combined monoglycerides and diglycerides or b) has a drop melting point measured by AOCS Standard Procedure Cc 18-80 of between 35 and 70 degrees C.

### CROSS-REFERENCE TO RELATED APPLICATIONS

This application claims the benefit of U.S. Provisional Application No. 63/367,352, filed Jun. 30, 2022, and U.S. Provisional Application No. 63/264,211, filed Nov. 17, 2021, each of which is incorporated by reference herein in its entirety.

### TECHNICAL FIELD

This application relates to natural oil-based petrolatum compositions, methods of making the same, and their use in personal care products.

### BACKGROUND

Petrolatum is a byproduct of petroleum refining. With a melting point close to body temperature, petrolatum softens upon application and forms an occlusive film around the applied area, thus creating an effective barrier against the evaporation of the skin's natural moisture and foreign particles or microorganisms that may cause infection. Petrolatum is odorless and colorless, and it has an inherently long shelf life, however, it is not readily biodegradable. Petrolatum is not a single entity but rather comprised of a complex mixture of organic compounds with a diversity of structures. This diversity of components allows petrolatum to have unique rheological properties over a wide variety of temperatures. For example, petrolatum does not have a distinct melting point like one traditionally thinks about in organic compounds, rather it melts over a temperature range and congeals at about the same temperature range. These properties make petrolatum a useful and popular ingredient in skincare products and cosmetics. It is often used as an ingredient in a wide variety of personal care products such as skin creams, lotions, hair care products, and cosmetics. A primary benefit is petrolatum's occlusive properties where it can create a barrier to protect or preserve hydration of the skin. Therefore, it is commonly used to protect skin, hair, and lips or to aid in the healing of damaged skin or lips. It is commonly known by the brand name Vaseline®.

When properly refined, petrolatum has no known health concerns. However, with an incomplete refining history, petrolatum could potentially be contaminated with polycyclic aromatic hydrocarbons, or PAHs. PAHs are byproducts of organic material combustion, commonly stored in fats upon exposure due to its lipophilic properties.

There have been numerous efforts to develop a bio-based alternative to petrolatum. Most of these efforts relate to creating blends of higher melting waxes, hydrogenated oils, or other natural oils. Through blending, it may be possible to create a product with a similar feel to petrolatum, however, these products suffer from a common disadvantage. Because they are simple blends, the rheology of the material does not match petrolatum through the range of application temperatures. These materials may have a polymodal melting profile where the lower melting components melt first, while higher melting components remain intact until the temperature reaches a higher point. Put another way, these substitute products do not have a smooth melting curve, or smooth change in rheology over a range of temperatures. Rather they have duel or multiple phased melting profiles so they do not mimic the performance of petrolatum over a variety of temperatures. In addition, these blends can have a much higher Iodine Value (IV) representing the presence of a significantly high degree of unsaturation in the natural oils. This degree of unsaturation is undesirable because it contributes to significantly lower oxidative stability over time. Finally, these substitute products can also have relatively high hydroxyl values. These high hydroxyl value products can be difficult to formulate into personal care applications because the hydrophilicity of the hydroxide groups creates additional surfactant effect that interferes with product formulations.

Accordingly, it would be advantageous to have improved natural based materials that more closely mimic the texture, appearance, morphology, rheology, stability, formulation and surfactant properties of petrolatum. It would be environmentally and economically desirable if such materials were more readily or completely biodegradable and derived from renewable raw materials, such as natural oils.

### SUMMARY

In contrast to the prior art's blends of ingredients, the compositions disclosed herein more closely mimic petroleum based petrolatum by containing a mixture of components with differing molecular weights and rheological properties. Creating such a product by blending would be exhaustively time consuming and costly.

The present disclosure relates to a composition comprising a triglyceride component wherein: the triglyceride component comprises a mixture of triglycerides and wherein the mixture of triglycerides comprises individual triglycerides comprising one or more ester containing fatty acids and wherein the esters of the ester containing fatty acids are C8-C22 branched or straight chain fatty acid esters and wherein the composition has a drop melting point measured by AOCS Standard Procedure Cc 18-80 of between 30° and 70° C.

The present disclosure provides a composition comprising a triglyceride component wherein: the triglyceride component comprises a mixture of triglycerides and wherein the mixture of triglycerides comprises individual triglycerides comprising one or more ester containing fatty acids and wherein the esters of the ester containing fatty acids are C8-C22 branched or straight chain fatty acid esters and wherein the composition contains less than 10% combined monoglycerides and diglycerides.

The present disclosure provides a composition comprising a triglyceride component wherein: the triglyceride component comprises a mixture of triglycerides and wherein the mixture of triglycerides comprises individual triglycerides comprising one or more ester containing fatty acids and wherein the esters of the ester containing fatty acids are C8-C22 branched or straight chain fatty acid esters and wherein the composition has: a) contains less than 10% combined monoglycerides and diglycerides, and b) has a drop melting point measured by AOCS Standard Procedure Cc 18-80 of between 30° and 70° C.

The low IV of the natural oil-based petrolatum disclosed herein leads to improved oxidative stability and correspondingly improved shelf life and quality. The lower hydroxyl value improves the ability of natural oil-based petrolatum disclosed herein to be utilized in personal care formulations more efficiently. In addition, the structure of the natural oil-based petrolatum disclosed herein is surprisingly biodegradable.

The natural oil-based petrolatum compositions described herein are useful for industrial applications and personal care products. In the case of personal care products specifically, it is desirable for the petrolatum substitute to have properties which can improve ease of manufacturing while providing a pleasing appearance and feel.

Advantages, some of which are unexpected, are achieved by aspects of the present disclosure. For example, various compositions described herein advantageously spread evenly and uniformly on the skin. They have a much more consistent rheology over a range of temperatures and more closely mimic the characteristics of petroleum-based petrolatum. The natural oil-based petrolatum compositions disclosed herein have an occlusive effect and the ability to coat and protect the skin from moisture loss.

The compositions of the present disclosure also has improved manufacturing properties and can be incorporated into personal care products such as shampoos, conditioners, creams, lotions, sun care, hair care, hair styling, body washes, and the like.

The compositions of the present disclosure also have distinct advantages over the prior art. In some applications, it can be advantageous for the compositions to have a low hydroxyl value to facilitate incorporation into finished products. Limiting the amount of MAGs and DAGs present in the compositions impacts the hydroxyl value and allows for ease of formulation into finished products.

In a separate aspect, the specific manufacturing process conditions limit the production of MAGs, DAGs, and associated free fatty acids. Limitation of free fatty acid production, particularly when castor oil or hydrogenated castor oil are utilized in the reaction, limits the creation of oligomers of hydroxy stearic acid as an additional product of the reaction. Any significant formation of these compounds contributes to undesirable rheology, corresponding production of MAGs and DAGS, and resistance to biodegradability.

As a further advantage, various compositions described herein are based on natural oils and thus have the advantage of comprising biodegradable, renewable, and environmentally-friendly components. For example, the natural oil-based petrolatum composition of the present disclosure can be prepared from natural oils and yet can offer the above-described advantages.

### DETAILED DESCRIPTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter. One aspect described in conjunction with a particular embodiment is not necessarily limited to that embodiment and can be practiced with any other embodiment(s).

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

As used herein, the singular forms “a,” “an,” and “the” and similar referents in the context of describing the elements (especially in the context of the following claims) include plural referents unless the context clearly dictates otherwise. For example, reference to “a substituent” encompasses a single substituent as well as two or more substituents, and the like. It is understood that any term in the singular may include its plural counterpart and vice versa, unless otherwise indicated herein or clearly contradicted by context.

The term “or” is used to refer to a nonexclusive “or” unless otherwise indicated. The statement “at least one of A and B” has the same meaning as “A, B, or A and B.”

In addition, it is to be understood that the phraseology or terminology employed herein, and not otherwise defined, is for the purpose of description only and not of limitation. Any use of section headings is intended to aid reading of the document and is not to be interpreted as limiting: information that is relevant to a section heading may occur within or outside of that particular section. To the extent allowed by law, any publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document: for irreconcilable inconsistencies, the usage in this document controls.

As used herein, the following terms have the following meanings unless expressly stated to the contrary.

As used herein, the terms “for example,” “for instance,” “such as,” or “including” are meant to introduce examples that further clarify more general subject matter. Unless otherwise specified, these examples are provided only as an aid for understanding the applications illustrated in the present disclosure and are not meant to be limiting in any fashion.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, plus or minus within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

As used herein, the term “natural oil” may refer to oil derived from plants or animal sources. The term “natural oil” includes natural oil derivatives, unless otherwise indicated. Examples of natural oils include, but are not limited to, vegetable oils, algae oils, animal fats, tall oils, derivatives of these oils, combinations of any of these oils, and the like. Representative non-limiting examples of vegetable oils include canola oil, rapeseed oil, coconut oil, corn oil, cottonseed oil, olive oil, palm oil, peanut oil, safflower oil, sesame oil, soybean oil, sunflower oil, linseed oil, palm kernel oil, tung oil, jatropha oil, mustard oil, camelina oil, pennycress oil, hemp oil, algal oil, jojoba oil, and castor oil. Representative non-limiting examples of animal fats include lard, tallow; poultry fat, yellow grease, and fish oil. Tall oils are by-products of wood pulp manufacture. In some aspects, the natural oil may be refined, bleached, and/or deodorized. In some aspects, the natural oil is present individually or as mixtures thereof.

As used herein, the term “hydrogenated” or “hydrogenated natural oil” refers to partial, complete, or substantially complete hydrogenation of a natural oil. Partial or substantially complete hydrogenation of natural oils is well known in the art. A skilled artisan will appreciate that is difficult and impractical to completely hydrogenate a natural oil as some unsaturation will most likely remain in any hydrogenated oil no matter the lengths taken during hydrogenation. Efforts to completely hydrogenate an oil will lead to economic inefficiencies and degradation of the oil. The extent of hydrogenation is typically reflected by reference to the products' residual iodine value. Therefore, many oils sold or referred to as “fully” hydrogenated have been processed to this point of diminishing returns and still have a small residual iodine value. Many hydrogenated natural oils may be purchased on the market and are available from a variety of commercial sources.

As used herein, a “natural oil-based” composition means that the composition contains oils and fatty acids which are predominantly, substantially or entirely, derived from natural oils and natural oil derivatives. The natural oil-based composition may, in various aspects, contain oils which are at least 50%, 55%, 60%, 65%, 70%, 75%, 80%, 85%, 90%, 95%, 99%, 99.9%, 99.99% or about 100% natural oil or hydrogenated natural oil.

A “monoacylglyceride” refers to a molecule having a glycerol moiety with a single fatty acid residue that is linked via an ester bond. The terms “monoacylglycerol,” “monoacylglyceride,” “monoglyceride,” and “MAG” are used interchangeably herein. Monoacylglycerides include 2-acylglycerides and 1-acylglycerides.

A “diacylglyceride” refers to a molecule having a glycerol moiety having two fatty acid residues linked via ester bonds. The terms “diacylglycerol,” “diacylglyceride,” “diglyceride,” and “DAG” are used interchangeably herein. Diacylglycerides include 1,2-diacylglycerides and 1,3-diacylglycerides.

A “triacylglyceride” refers to a molecule having a glycerol moiety that is linked to three fatty acid residues via ester bonds. The terms “triacylglycerol,” “triacylglyceride,” “triglyceride,” and “TAG” are used interchangeably herein. In some aspects the triglyceride is comprised of C8-C22 fatty acids. In further aspects the triglyceride comprises hydroxy containing fatty acids.

The hydroxy containing fatty acids of a triglyceride may be further modified by esterification. The hydroxy containing fatty acid can be reacted with a free fatty acid to create an ester bond and therefore correspondingly an ester containing fatty acid. In some aspects the triglyceride comprises ester containing fatty acids. In some aspects, more than 20 percent of the hydroxy containing fatty acids are esterified. In some aspects, more than 30 percent of the hydroxy containing fatty acids are esterified. In some aspects, more than 40 percent of the hydroxy containing fatty acids are esterified. In some aspects, more than 50 percent of the hydroxy containing fatty acids are esterified. In some aspects, more than 20 percent of the triglyceride fatty acids are substituted with a C8-C22 fatty acid ester. In some aspects, more than 30 percent of the triglyceride fatty acids are substituted with a C8-C22 fatty acid ester. In some aspects, more than 40 percent of the triglyceride fatty acids are substituted with a C8-C22 fatty acid ester. In some aspects, more than 50 percent of the triglyceride fatty acids are substituted with a C8-C22 fatty acid ester. In some aspects, between 20 percent and 90 percent of the triglyceride fatty acids are substituted with a C8-C22 fatty acid ester. In some aspects, between 20 percent and 70 percent of the triglyceride fatty acids are substituted with a C8-C22 fatty acid ester. In some aspects, between 30 percent and 50 percent of the triglyceride fatty acids are substituted with a C8-C22 fatty acid ester.

The term “fatty acid” as used herein can refer to a molecule comprising a hydrocarbon chain and a terminal carboxylic acid group. As used herein, the carboxylic acid group of the fatty acid may be modified or esterified, for example as occurs when the fatty acid is incorporated into a glyceride or another molecule (e.g., COOR, where R refers to, for example, a hydrocarbon chain). Alternatively, the carboxylic acid group may be in the free fatty acid or salt form (i.e., COO″ or COOH). The ‘tail’ or hydrocarbon chain of a fatty acid may also be referred to as a fatty acid chain, fatty acid sidechain, or fatty chain whether it is in its esterified or free form. The hydrocarbon chain of a fatty acid will typically be a saturated or unsaturated aliphatic group. A fatty acid having N number of carbons, will typically have a fatty acid side chain having N−1 carbons.

The subject application also relates to modified forms of fatty acids and thus the term fatty acid may be used in a context in which the fatty acid has been substituted or otherwise modified as described. For example, in various aspects, a fatty acid may be substituted with another alkyl chain (as is the case for isostearic acid or a hydroxy group as is the case with ricinoleic acid present in castor oil.)

An “acylglyceride” refers to a molecule having at least one glycerol moiety with at least one fatty acid residue that is linked via an ester bond. For example, acylglycerides can include monoacylglycerides, diacylglycerides, triacylglycerides. The group acylglycerides can be further refined by additional descriptive terms and can be modified to expressly exclude or include certain subsets of acylglycerides. For example, the phrase mono- and di-acylglycerides refers to MAGs (monoacylglycerides) and DAGs (diacylglycerides), while the phrase non-MAG/non-DAG acylglycerides refers to a group of acylglycerides which exclude MAGs and DAGs.

Fatty acids and/or the natural oils containing them may be hydrogenated as described herein.

The levels of particular types of fatty acids may be provided herein in percentages out of the total fatty acid content of an oil. Unless specifically noted otherwise, such percentages are weight percentages based on the total fatty acids, including free fatty acids and esterified fatty acids as calculated experimentally by methods well known to the skilled artisan.

A “saturated” fatty acid is a fatty acid that does not contain any carbon-carbon double bonds in the hydrocarbon chain. An “unsaturated” fatty acid contains one or more carbon-carbon double bonds. A “polyunsaturated” fatty acid contains more than one such carbon-carbon double bond while a “monounsaturated” fatty acid contains only one carbon-carbon double bond. Carbon-carbon double bonds may be in one of two stereoconfigurations denoted cis and trans. Naturally-occurring unsaturated fatty acids are generally in the “cis” form.

Non-limiting examples of fatty acids include C8, C10, C12, C14, C16 (e.g., C16:0, C16:1), C18 (e.g., C18:0, C18:1, C18:2, C18:3, C18:4), C20 and C22 fatty acids. For example, the fatty acids can be caprylic (8:0), capric (10:0), lauric (12:0), myristic (14:0), palmitic (16:0), stearic or isostearic (18:0), oleic (18:1), linoleic (18:2) and linolenic (18:3) acids.

The term “C8-C22 fatty acid” means a fatty acid containing 8-22 carbons. The C8-C22 fatty acid may be straight or branched and may be substituted with additional substituent groups such as a C1-C3 alkyl group, a hydroxyl group, or an ester group. In some aspects the C8-C22 fatty acid has a straight chain. In some aspects, the C8-C22 fatty acid is a C16 or C18 fatty acid. In some aspects, the C8-C22 fatty acid comprises stearic acid. In some aspects, the C8-C22 fatty acid comprises greater than 40% or greater than 70% stearic acid. In some aspects, the C8-C22 fatty acid comprises between 40% and 95% stearic acid.

The C8-C22 fatty acid may be a mixture of C8-C22 fatty acids. Stearic acid is commercially available in a variety of purities. It may be sold as 1890, meaning 90% C18 (stearic) containing. The remainder is typically comprised of other fatty acids, predominately C16. Alternatively stearic can be sold as 1845 (or 1655); meaning approximately 45% stearic and 55% palmitic. Inn some aspects, the C8-C22 fatty acid consists essentially of stearic and palmitic acid.

In any aspect, the C1-C3 alkyl substituent may be selected from methyl, ethyl, or propyl. In any embodiment, the C1-C3 alkyl substituent may be methyl. The C8-C22 fatty acid substituted with one or more C1-C3 alkyl substituents, in any embodiment described herein, may be isopalmitic acid, isomyristic acid, isosteric acid, 19-methylarachidic acid, isolauric acid.

The term “isostearic acid” as used herein refers to the chemical 16-methylheptadecanoic acid, which is a methyl-branched fatty acid that is heptadecanoic acid substituted by a methyl group at position 16. Isostearic acid is a lightly-branched, liquid fatty acid which can be produced by the reaction of oleic acid with a natural mineral catalyst. Isosteric acid is used in applications which require a liquid fatty acid with stability: thermal stability in the case of a lubricant, odor stability for a cosmetic formulation, and oxidation stability for products with long shelf-life requirements. The branching structure of isostearic acid also enhances its dispersing power, and it is used in cosmetic and industrial applications for the stabilization of pigments and mineral particles in oils and solvents. Isosteric acid is well known and commercially available. As used here in the term isosteric acid refers to a composition that comprises substantially all isosteric acid but need not be 100% pure. The term isosteric acid also specifically includes all potential isomers of isosteric acid where the methyl substituent occurs at various locations on the fatty acid chain.

The fatty acid composition of an oil can be determined by methods well known in the art. The American Oil Chemist's Society (AOCS) maintains analytical methods for a wide variety of tests performed on vegetable oils. Hydrolysis of the oil's components to produce free fatty acids, conversion of the free fatty acids to methyl esters, and analysis by gas-liquid chromatography (GLC) is the universally accepted standard method to determine the fatty acid composition of an oil sample. The AOCS Procedure Ce 1-62 describes the procedure used.

The terms “esterification or esterified” means the creation of an ester bond including: 1) the dehydration reaction of an alcohol with an acid: 2) transesterification, the reaction of an alcohol with an ester to form a new ester; or 3) interesterification, the rearrangement of fatty acids within a triacylglycerol structure.

A “drop point” or “dropping point” generally refers to the temperature at which a material (such as a wax) softens and becomes sufficiently fluid to flow as determined under the conditions of a given standardized test. As used herein, drop points are determined via AOCS Standard Procedure Cc 18-80. (Official Methods and Recommended Practices of the American Oil Chemists' Society, 7th Edition). Drop point is similar to melting point in that it reflects the thermal characteristics of a compound, however, drop point can be useful in defining materials which do not have a defined melting point. In some aspects, the natural oil-based petrolatum exhibits a drop melt point of about 30° C. to about 70° C. In some aspects, the natural oil-based petrolatum exhibits a drop melt point of about 35° C. to about 50° C.

The term “Polydispersity Index” (also known as “Molecular Weight Distribution”) as used herein is the ratio of weight average molecular weight (Mw) to number average molecular weight (Mn). The polydispersity data is collected using a Gel Permeation Chromatography instrument equipped with a Waters 510 pump and a 410 differential refractometer. Samples are prepared at an approximate 2% concentration in a THF solvent. A flow rate of 1 ml/minute and a temperature of 35° C. are used. The columns consist of a Phenogel 5 micron linear/mixed Guard column, and 300×7.8 mm Phenogel 5 micron columns (styrene-divinylbenzene copolymer) at 50, 100, 1000, and 10000 Angstroms. Molecular weights were determined using the following standards:

| Standard | Mono-olein | Diolein | Arcol LHT 240 | Trio-lein |
|---|---|---|---|---|
| Mol. | 356 | 620 | 707 | 878 |
| Weight | | | | |
| (Daltons) | | | | |

| | Epoxidized | | | Acclaim |
|---|---|---|---|---|
| Standard | Soybean Oil | Acclaim 2200 | Mult-ranol 3400 | 8200 |
| Mol. | 950 | 2000 | 3000 | 8000 |
| Weight | | | | |
| (Daltons) | | | | |

The term “weight average molecular weight” as used herein refers to M_{w}, which is equal to ΣMᵢ²nᵢ/ΣMᵢnᵢ, where nᵢ is the number of molecules of molecular weight Mᵢ. In various examples, the weight-average molecular weight can be determined using the test described herein or through size exclusion chromatography, light scattering, small angle neutron scattering, X-ray scattering, and sedimentation velocity.

The term “number average molecular weight” as used herein refers to Mₙ, which is equal to the total weight of the sample divided by the number of molecules in the sample. Mₙ, can be represented by the formula ΣMᵢnᵢ/nᵢ, where nᵢ is the number of molecules of molecular weight Mᵢ.

In some aspects, the natural oil-based petrolatum exhibits a polydispersity index of greater than 1.3. In some aspects, the natural oil-based petrolatum exhibits a polydispersity index of between 1.3 and 2.0.

The term “Acid Value” (AV) as used herein is defined as the weight of KOH in mg needed to neutralize the organic acids present in 1 g of test sample and it is a measure of the free fatty acids present in the composition. AV can be determined by the AOCS Official Method Cd 3d-63. The acid value of the compositions described herein may be less than 20.0, or less than 10.0, or less than 4.0, or between 0.5 and 20.0, or between 0.5 and 10.0, or between 0.5 and 4.0.

The term “Hydroxyl Value” as used herein is expressed in milligrams of potassium hydroxide and corresponds to the number of hydroxyl groups present in 1 g of a sample, is one of the traditional characteristics of oils and fats. Hydroxyl Value may be determined by AOCS Standard Method Cd 13-60. The compositions described herein may have a hydroxyl value of less than 90 or less than 50. In some aspects, the composition may have a hydroxyl value of between 10 and 90 or between 30 and 90. In some aspects, the composition may have a hydroxyl value of between 50 and 90.

The term “Iodine Value” (commonly abbreviated as IV) as used herein is the mass of iodine in grams that is consumed by 100 grams of a chemical substance. Iodine numbers are often used to determine the amount of unsaturation in fats, oils and waxes. In fatty acids, unsaturation occurs mainly as double bonds which are very reactive towards halogens, iodine in this case. Thus, the higher the iodine value, the more unsaturation is present in the sample. The Iodine Value of a material can be determined by the standard well-known Wijs method (A.O.C.S. Cd1-25).

### Natural Oil-Based Petrolatum Composition

The natural oil-based petrolatum composition described herein has a unique composition which provides a more consistent rheology over a variety of temperatures more closely mimicking petroleum-based petrolatum.

The present disclosure provides a personal care composition comprising a triglyceride component wherein: the triglyceride component comprises a mixture of triglycerides and wherein the mixture of triglycerides comprises individual triglycerides comprising one or more ester containing fatty acids and wherein the esters of the ester containing fatty acids are C8-C22 branched or straight chain fatty acid esters and wherein the composition has a drop melting point measured by AOCS Standard Procedure Cc 18-80 of between 30° and 70° C.

The present disclosure provides a personal care composition comprising a triglyceride component wherein: the triglyceride component comprises a mixture of triglycerides and wherein the mixture of triglycerides comprises individual triglycerides comprising one or more ester containing fatty acids and wherein the esters of the ester containing fatty acids are C8-C22 branched or straight chain fatty acid esters and wherein the composition contains less than 10% combined monoglycerides and diglycerides. The triglyceride component may be prepared by the skilled artisan for example by epoxidizing natural oils containing unsaturated fatty acids and ring opening the epoxides. This chemistry is well known in the fats and oils art. Alternatively, the triglyceride component may naturally contain hydroxy groups. Some natural oils contain hydroxy fatty acids in their native state. Castor oil is one such example. Typically, castor oil is comprised of approximately 70%-90% ricinoleic acid fatty acid residues. The triglyceride component may be partially, substantially, or completely hydrogenated. Good quality castor oil has a hydroxyl value of approximately 160. Fully hardened or hydrogenated castor oil typically has a minimal hydroxyl value of 150.

Procedures of the present disclosure are tailored to minimize the amount of transesterification and interesterification that occurs during the reaction. Excessive transesterification can create hydroxystearic oligomers and high molecular weight structures as well as unwanted MAGs and DAGs. In some aspects the compositions contain less than 10% combined MAGs and DAGs. In some aspects the compositions contain less than 10% combined MAGs and DAGs. In some aspects the compositions contain between 0.5% and 10% combined MAGs and DAGs. In some aspects the compositions contain between 1% and 8% combined MAGs and DAGs. The content of MAGs and DAGs in the composition can be determined routinely by those of skill in the art. Size exclusion chromatography or GPC as described above can be used to determine molecular weight and correspondingly fractions of a composition that are mono, di, or triglycerides. A skilled artisan will appreciated that a standard curve can be created and used to celebrate the specific chromatography equipment.

In some aspects, the triglyceride component is hydrogenated. In some aspects the triglyceride component comprises hydrogenated castor oil.

In some aspects, the reaction mixture comprises and additional natural or hydrogenated natural oil.

In some aspects, the additional natural oil is hydrogenated soy bean or hydrogenated coconut oil.

The composition may include minimal amounts of free fatty acids. For example, the composition may include less than about 2 wt % free fatty acids. In another embodiment, the composition may include less than about 1 wt %, less than about 2.5 wt % free fatty acids, or between 0.1 wt % and 2.5 wt % fatty acids.

The composition may include minimal amounts of combined monoglycerides and diglycerides. For example, the composition may include less than about 10 wt % of combined monoglycerides and diglycerides. In another aspect, the composition may include less than about 8 wt %, about 6 wt %, less than about 5 wt %, or less than about 3 wt % of combined monoglycerides and diglycerides. In another aspect, the composition may include between about 1% to about 10 wt %; or between about 1% to about 7 wt %; or between about 2% to about 5 wt %; or between 1% to about 4%, of combined monoglycerides and diglycerides.

The iodine value of the compositions described herein may be less than about 5.0, or less than about 3.0, or in between about 0.1 to about 3.

The compositions, as described herein in any embodiment, may include one or more of the following: (i) an acid value of less than about 20.0; (ii) between about 2% to about 7 wt %; of combined monoglycerides and diglycerides, or (iii) an iodine value of less than about 10.0. In some aspects the natural oil-based-petrolatum composition may have two, or all three, of the preceding characteristics.

Unlike waxes or hard fats, the natural oil-based petrolatum formulations described herein can be a semisolid material that can hold its own shape but deflects under pressure more similar to a grease or shortening. Resistance to deflection under pressure can be determined though use of a cone penetration test. Cone penetration can be measured by use of standard methodology ASTM D217-2.

The natural oil-based petrolatum exhibits a combination of rheological properties that provides for comparable spreading and tackiness to petroleum-based petrolatum. In any embodiment disclosed herein, the natural oil-based petrolatum exhibits one or more rheological properties selected from a melt drop point of about 35° C. to about 70° C., a cone penetration at 25° C. of greater than 20 or from about 20 to about 100 or from about 60 to about 90 (Dmm (1/10 of mm), kinetic viscosity at 100° C. of about 5 mm²/s to about 35 mm²/s, a congealing point of about 25° C. to about 45° C., or combinations thereof.

### Method of Preparing Natural Oil-Based Petrolatum Composition (Also Referred to Herein Simply as Compositions)

The present disclosure also provides a method of making a natural oil-based petrolatum composition. The method involves mixing a fatty acid and a triglyceride component containing a one or more hydroxyl containing fatty acid chains and optionally a hydrogenated natural oil, heating the mixture to an elevated temperature, (optionally in the presence of an acid catalyst) and exposing the heated mixture to pressure below ambient pressure to yield a product wherein one or more of the hydroxyl containing fatty acid chains are esterified with a C8-C22 branched or straight chain fatty acid and wherein the triglyceride component: a) contains less than 10% combined monoglycerides and diglycerides and/or b) has a drop melting point measured by AOCS Standard Procedure Cc 18-80 of between 30° and 70° C. and isolating the petrolatum composition.

The reaction may be monitored in a number of different ways depending on the properties sought. If allowed to proceed the reaction will reach a certain steady state point where a form of equilibrium is achieved. At this point the parameters of the product will not be changing dramatically and continued reaction time will encourage degradation to affect the quality of the product. Alternatively, the reaction may be allowed to proceed to a certain set point such as acid value, hydroxyl value, or until a certain drop melting point is achieved. This is in the discretion of the operator. In some aspects the reaction is allowed to proceed until the reaction mixture reaches an acid value of less than 20.0, or less than 10, or less than 5, or until the reaction mixtures reaches an acid value of less 4.0 so as to provide a natural oil-based petrolatum composition. In some aspects that reaction mixture reaches an acid value between 0.5 and 20.0. In some aspects that reaction mixture reaches an acid value between 0.5 and 10.

The reaction mixture has the composition described herein and the mixture is treated to induce chemical or enzymatic esterification by methods well known in the art. Procedures of the present disclosure, including use of vacuum and limited catalysts, are tailored to minimize the amount of transesterification and interesterification that occurs during the reaction. Excessive transesterification an create hydroxystearic oligomers and high molecular weight structures which lower biodegradability as well as unwanted MAGs and DAGs.

To carry out chemical esterification, a catalyst can be added at an amount of about 0.1 wt % relative to the reaction mixture of ingredients. Example catalysts can be acids such methanesulfonic acid or bases such as sodium hydroxide and calcium hydroxide, or metal catalysts. In some aspects, methanesulfonic acid is the catalyst. Hypophosphoric acid can optionally be added to the reaction mixture to prevent formation of off colors. The reaction temperature can then be increased to about 140°−250° C. Typically, a reaction temperature of approximately 160° C. is utilized. This reaction temperature is maintained for a period of time and the reaction vessel is subjected to vacuum to achieve a pressure of between 20 and 50 torr until a desired endpoint or steady state is reached. In some aspects to an acid value of less than 15 or less than 10 or less than 5 is achieved or a polydispersity index of greater than 1.3 is obtained. A base, for example a mineral base such as sodium hydroxide or calcium hydroxide, can be added at an amount of about 0.2 wt % to neutralize the catalyst with a slight excess. The reaction mixture can then be cooled to a temperature ranging from about 60°−80° C. A filter media, for example acid activated beaching clay such as B80 neutral or Trisyl® silica, can be added to the reaction mixture in an amount of about 2 wt % or less relative to the reaction mixture to remove impurities. The final product, i.e., the natural oil-based petrolatum composition, is then filtered to remove the salts, silica, or clay mixture.

Alternatively, to carry out enzymatic transesterification, an enzymatic catalyst can be added at an amount of 2 wt % relative to the reaction mixture. An example enzymatic catalyst can be Lipase Novozyme 435. A vacuum of about 50 torr can be used to remove water as the reaction is taking place. A reaction temperature ranging from about 60-80° C. is maintained until an acid value of less than 5.0 is achieved or a polydispersity index of greater than 1.3 is obtained. The enzymatic catalyst can then be filtered out using an appropriate filter device to obtain the final product, i.e., the natural oil-based petrolatum composition.

Alternatively, esterification can be performed without a catalyst. A reaction mixture of a C8-C22 branched or straight chain fatty acid and a hydrogenated natural oil such as castor oil, are pre-melted and heated to a temperature ranging from 60°−80° C. before adding to a reaction vessel along with a nitrogen sparge to prevent oxidation. A vacuum is applied to the reaction vessel to achieve a pressure of between 20 and 50 torr and the temperature increased to 180°−250° C.

Lower pressures and/or temperatures may also be utilized depending on the equipment chosen for the reaction. The acid value of the reaction is monitored, and the reaction temperature and vacuum are maintained until an acid value of less than 20 (or other endpoint) is achieved. The product is isolated after cooling the reaction mixture to 60°−80° C. and filtering (such as through a sock filter) to remove any particulates.

### Topical Formulation

The compositions described herein may be utilized in a wide variety of personal care products. It has desirable melting and spreading properties as well as the ability to provide occulsivity to the skin. In addition, it is used in soaps, body washes, and hair products to provide a desirable texture and feel after use. The compositions described here in can substitute for petroleum-based petrolatum in most or all of these applications. The skilled artisan may need to slightly modify or tailor the personal care formula to adapt it slightly to take advantage of the quality (or ameliorate any undesired affect) of the natural based petrolatum disclosed herein. This kind of adaptation is well understood in the art and is undertaken for any new ingredient or even for known ingredients coming from a different supplier.

The inventors unexpectedly found that formulations comprising a these compositions have numerous desirable characteristics as explained further below and can be used to replace all or part of the petroleum based petrolatum currently used in personal care or cosmetic formulations.

In one aspect, the present invention is a topical formulation comprising a natural oil-based petrolatum composition as described herein. As used herein, the term “topical formulation” refers to a formulation that may be applied directly to a part of the body. The term “formulation” is used herein to denote compositions of various ingredients in various weight ranges, in accordance with the present disclosure for use in personal or home care.

“Personal care” means and comprises any cosmetic, hygienic, toiletry and topical care products including, without limitation, leave-on products (i.e., products that are left on the skin or keratinous substrates after application): rinse-off products (i.e., products that are washed or rinsed from the skin and keratinous substrates during or within a few minutes of application); shampoos; hair curling and hair straightening products; combing or detangling creams, hair style maintaining and hair conditioning products (either concentrated masks or more standard formulations; whether rinse-off or leave-on); lotions and creams for nails, hands, feet, face, scalp and/or body; hair dye; face and body makeup; foundation; masks; nail care products; astringents; deodorants; antiperspirants; anti-acne; antiaging; depilatories; colognes and perfumes; skin protective creams and lotions (such as sunscreens); skin and body cleansers/body washes; face cleansers; skin conditioners; skin toners; skin firming compositions; skin tanning and lightening compositions; liquid soaps; bar soaps; syndet bars; bath products; shaving products; personal lubricants, and oral hygiene products (such as toothpastes, oral suspensions, and mouth care products).

The natural oil-based petrolatums disclosed herein can be utilized alone on the skin or hair and are particularly useful in reducing or replacing the various components in shampoos, body washes, hair care, detangling, and conditioner formulations.

The natural base petrolatum disclosed here in is particularly useful in the preparation of emulsions in personal care product (including very small droplet size emulsions such as microemulsion or even nanoemulsions). Many of the personal care formulations such as lotions, sunscreens, body washes, and hair care products are based on oil in water emulsion technology. Preparation of these emulsions is well known in the art. Examples of such technology can be found in the following published patent documents. WO/2006/024095; EP3743038; and EP1572333.

The natural base petrolatum disclosed here in is particularly useful in the preparation of products for the lubrication of the skin such as personal hygiene products, diaper care and anti-chaffing products, shaving preparations, and preparations for sexual activities.

The hydroxyl value of the natural based petrolatum by be important to forming a stable emulsion. If the hydroxyl value is too high it may disrupt the formulation or interfere with stability. A skilled artisan would appreciate that some degree of hydroxyl value may beneficially contribute to overall surfactancy and the formulation and other corresponding surfactants and/or emulsifiers may be adjusted to compensate.

The texture of such personal care formulations is not limited and may be, without limitation, a liquid, gel, spray, emulsion (such as lotions and creams), shampoo, conditioner, combing cream, pomade, foam, tablet, stick (such as lip care products), makeup, suppositories, among others, any of which can be applied to the skin or hair and which typically are designed to remain in contact therewith until removed, such as by rinsing with water or washing with shampoo or soap or syndet bars. Other forms could be gels that can be soft, stiff, or squeezable. Sprays can be non-pressurized aerosols delivered from manually pumped finger-actuated sprayers or can be pressurized aerosols such as mousse, spray, or foam forming formulation, where a chemical or gaseous propellant is used.

Formulations prepared using the natural oil-based petrolatum disclosed herein have a white or pale white color that is generally considered to be aesthetically appealing. In some cases, the formulations of this disclosure may be further processed to make a colored end product. In such cases, the white color is beneficial because it will show up the additional pigment without influencing the final color.

Formulations containing the natural oil-based petrolatum of the present disclosure may optionally contain additional ingredients to tailor the viscosity to the needs of the particular application. A skilled artisan will readily appreciate the range of additives available to suit this purpose including but not limited to the following: sclerotium gum, xanthan gum, carrageenan, gellan gum, native starches, modified starches, sodium starch octenyl succinate, aluminum starch succinate, hydroxypropyl starch phosphate, pectin, calcium citrate, salt(s) NaCl, KCl, acrylate polymers, acrylate based copolymers, carbomers, cellulose, citrus fibres and derivatives, hydroxy ethyl cellulose, carboxy methyl cellulose, polyols such as sorbitol, and mixtures thereof. These additives may be utilized to add texture, viscosity, or structure to the formulations. A skilled artisan would appreciate that these ingredients may be present in various concentrations depending on the needs of the particular formulation and may even be the predominant element of a particular formulation.

Formulations containing the natural oil-based petrolatum of the present disclosure may optionally contain at least one further ingredient chosen from the group consisting of preservative, salt, vitamin, emulsifier, texturizer, nutrient, micronutrient, sugar, protein, polysaccharide, polyol, glucose, sucrose, glycerol, sorbitol, pH adjusters, emollients, dyes, pigments, skin actives, oils, hydrogenated oils, waxes, or silicones.

Formulations containing the natural oil-based petrolatum of the present disclosure may have a wide range of pH values. Aspects of this disclosure include formulations having pH between 3-11, or between 4-8, or between 4-7.

Formulations of the present disclosure may contain any useful amount of the natural oil-based petrolatum of the present disclosure. Formulations will preferably contain between 1%-100%, 50%-99%, 75%-95%, 20%-90%, 20%-80%, 1%-30%, 2%-20%, 3%-5% or 1%-8% by weight natural oil-based petrolatum in the final formulations. For example, the natural oil based petrolatum may be 90%-100% of the formulation as a skin protectant or ointment to replace Vasoline. In lip care products such moisturizers or cosmetic colorants, the natural oil based petrolatum may be 5%-70% of the formulation. In skin cream, facial creams, or night creams, the natural oil based petrolatum may be 5%-30% of the formulation. In body lotions, the natural oil based petrolatum may be 10%-30% of the formulation. In oral care products such as toothpaste, the natural oil based petrolatum may be 1%-50% of the formulation. In body wash or liquid soaps, the natural oil based petrolatum may be 3%-10% of the formulation. In hard hair styling products, the natural oil based petrolatum may be 20%-50% of the formulation. In softer hair styling formulations such as spray or sprays or lotions, the natural oil based petrolatum may be 1%-5% of the formulation. In hair conditioners, shampoos, or 2 in 1 shampoo/conditioner formulations, the natural oil based petrolatum may be 1%-5% of the formulation. In hair mask applications, the natural oil based petrolatum may be 2%-5% of the formulation. A person of skill in the art would be able to adjust the level of inclusion to achieve the desired result.

The natural oil based petrolatum disclosed here in is particularly useful in the preparation of skin cleansing agent and wet skin treatments. Examples of such technology can be found in the following published patent documents. U.S. Pat. No. 9,943,468: US20170049673; JP5759389: U.S. Pat. Nos. 8,263,538; 8,134,054; and U.S. Pat. No. 6,716,440.

The natural oil based petrolatum disclosed here in is particularly useful in the preparation of moisturizing lotions. Examples of such technology can be found in the following published patent documents. U.S. Pat. No. 8,840,911 and EP322088.

The natural oil based petrolatum disclosed here in is particularly useful in the preparation of oral care and teeth whitening. Examples of such technology can be found in the following published patent document. U.S. Pat. No. 3,936,194

The natural oil based petrolatum disclosed here in is particularly useful in the preparation of sunscreen formulation. Examples of such technology can be found in the following published patent document. U.S. Pat. No. 10,375,952

The natural oil based petrolatum disclosed here in is particularly useful in the preparation of shampoos. Examples of such technology can be found in the following published patent document. JP5694187

The natural oil based petrolatum disclosed here in is particularly useful in the preparation of cosmetics and as a carrier of active ingredients. Examples of such technology can be found in the following published patent documents. U.S. Pat. Nos. 9,668,957 and 9,682,021.

The natural oil based petrolatum disclosed here in is particularly useful in the preparation of lip balms and cosmetic lip sticks. Examples of such technology can be found in the following published patent document. U.S. Pat. No. 7,776,347

All cited patent documents show some aspect of the current state of the art and are hereby incorporated by reference to the extent allowed.

In some aspects the personal product comprising the natural oil-based petrolatum is a body wash, face wash, shampoo, conditioner, combing cream, leave-on conditioner, skin moisturizer, lip moisturizer, or cosmetic.

In another embodiment, the composition is the esterification product of: about 55 wt % to about 85 wt % of a fully hydrogenated castor oil, about 15 wt % to about 45 wt % a C8-C22 branched or straight chain fatty acid.

In another embodiment, the composition is the esterification product of: about 55 wt % to about 85 wt % of a fully hydrogenated castor oil, about 15 wt % to about 45 wt % a C8-C22 branched or straight chain fatty acid and about 5 wt % to about 15 wt % a hydrogenated natural oil other than castor oil.

Any and every combination of two or more features disclosed herein for the natural oil-based petrolatums has been specifically contemplated and envisioned by the inventors. Therefore, the inventors have conceived of, and accordingly disclosed, every combination of single points and ranges disclosed for the triglyceride component containing a plurality hydroxyl containing fatty acid chains and the C8-C22 branched or straight chain fatty acid; as well as each and every combination of one or more of the value or ranges of the following parameters: drop melting point, cone penetration, congealing point, hydroxyl value, acid value, iodine value, and polydispersity index.

TABLE 1
EXAMPLES

| Materials | Source |
|---|---|
| Stearic Acid (TRV1890) | Twin Rivers Technology |
| Stearic Acid (TRV 1655) | |
| Hydrogenated Castor Oil (HCO) | Acme Hardesty |
| Hydrogenated Soybean Oil (HSO) | Cargill Incorporated |

TABLE 2

| Example | 1A | 2A | 2B | 2C | 2D | 1B |
|---|---|---|---|---|---|---|
| | Acid | No | No | No | No | Acid |
| | catalyzed | catalyst | catalyst | catalyst | catalyst | catalyzed |
| HCO* | 81 | 76 | 71 | 64 | 66 | 61 |
| Stearic | 19 | 24 | 29 | 36 | 34 | 29 |
| Acid 1890* | | | | | | |
| HSO* | — | — | — | — | — | 10 |
| OH Value | 85 | 85 | 75 | 47 | 53 | 34 |
| Acid Value | 2 | 7 | 17 | 18 | 17 | 4 |
| MDP ° C. | 33.55 | 60.4 | 61 | 40.17 | 47.78 | 36.8 |
| % MAG | 3.93 | 1.38 | 1.78 | 1.21 | 0.97 | 1.34 |
| % DAG | 1.64 | 0.87 | 1.61 | 1.69 | 3.46 | 6.04 |

*Amounts of HCO, Stearic acid, and HSO are % by weight of the reaction mixture.

### Example 1

The following chemical esterification method was carried out to make Examples 1A and 1B. All components or oils as described in Table 2 were pre-melted and heated to 80° C. before adding to the reaction vessel under a nitrogen sparge. The agitator was turned on to mix the contents. Hypophosphoric acid was added at 0.2% dosage and methanesulphonic acid was added at 0.1% dosage. Once all ingredients were added and well mixed the temperature was increased to approximately 160° C. Acid value is monitored throughout the reaction and once AV is <20 or change in AV is less than 1 unit per hour, vacuum is applied to the reaction vessel to achieve a pressure of approximately 30 torr. The reaction temperature was maintained until an acid value of 5 or less was achieved. The reaction was then cooled to approximately 85° C. and calcium hydroxide solution was added to neutralize the catalyst with a slight excess. The mixture was cooled to 70° C. and Trisyl® silica was added to the reaction at 1% and allowed to absorb the salts from the catalyst. The product was then filtered to remove the salts and clay mixture as well as other impurities.

### Example 2

The following chemical esterification method was carried out to purposefully limit transesterification in preparation of Examples 2A-2D. All components or oils as described in Table 2 were pre-melted and heated to 80° C. before adding to the reaction vessel under a nitrogen sparge to keep the product from oxidizing during the reaction. The agitator was turned on to mix the contents. Hypophosphoric acid was added at 0.2% dosage. Once all ingredients were added and well mixed the temperature was increased to approximately 180° C. and vacuum is applied to the reaction vessel to achieve a pressure of approximately 20-30 torr. Acid value is monitored throughout the reaction. The reaction temperature and vacuum were maintained until an acid value of 20 or less was achieved. The reaction was then cooled to approximately 85° C. and filtered through a sock filter to remove any particulates.

### Example 3

TABLE 3

| Example | 3A | 3B | 3C | 3D | 3E | 3F | 3H | 3I | 3J | 3K |
|---|---|---|---|---|---|---|---|---|---|---|
| | No | No | Acid | No | Acid | No | No | Acid | Acid | No |
| | catalyst | catalyst | catalyst | catalyst | catalyst | catalyst | catalyst | catalyst | catalyst | catalyst |
| Scale (g) | 1350 | 1400 | 1400 | 75500 | 1400 | 1400 | 1400 | 600 | 1350 | 3525 |
| Run Time (hrs) | 28 | 26 | 11 | 14 | 12 | 11 | 16 | 9 | 6 | 13 |
| HCO* | 80% | 66% | 66% | 66% | 66% | 71% | 64% | 81% | 78% | 78% |
| Stearic | 20 | | | | | | | | | 22% |
| Acid 1655* | | | | | | | | | | |
| Stearic | | 34% | 34% | 34% | 34% | 29% | 36% | 19% | 22% | |
| Acid 1890* | | | | | | | | | | |
| OH Value | 63.471 | 46.9 | 27.6 | 53.2 | 55.0 | 75.1 | 47.4 | 84.6 | 76.8 | 84.2 |
| Acid Value | 1.24 | 7.4 | 2.9 | 17.3 | 17.6 | 17.0 | 17.5 | 2.1 | 2.2 | 4.9 |
| MDP ° C. | 38.1 | 34.1 | 35.4 | 47.8 | 33.4 | 61.0 | 40.2 | 33.6 | 57.6 | 52.5 |
| RXN comments | ** | ** | ** | *** | *** | ** | ** | ** | ** | ** |

*Amounts of HCO and Stearic acid are % by weight of the reaction mixture.
** Reaction is run until essentially completion or equilibrium where values are not changing dramatically.
*** Reaction is run until a certain AV is achieved.

Examples 3A-K were prepared as represented in Table 2. They were run at different scales, for different times and utilized either a catalyst free or acid catalyst system as described in Examples 1 and 2. The reaction was allowed to either continue until a stable state where equilibrium is reached or was run until an acid value of less that 18 was reached.

### Example 4

TABLE 4

| Example | 4A | 4B | 4C | 4D | 4E | 4F |
|---|---|---|---|---|---|---|
| | No | Acid | No | Acid | Acid | Acid |
| | catalyst | catalyst | catalyst | catalyst | catalyst | catalyst |
| Scale (g) | 500 | 600 | 3500 | 1360 | 1350 | 1350 |
| Run Time (hrs) | 27 | 7 | 12.5 | 6 | 8 | 11 |
| HCO* | 76% | 76% | 77% | 76% | 76% | 76% |
| Stearic | 24% | 24% | 23% | 24% | 24% | 24% |
| Acid 1890* | | | | | | |
| OH Value | 83.0 | 73.1 | 75.6 | 67.7 | 68.0 | 59.0 |
| Acid Value | 7.2 | 2.4 | 4.8 | 2.5 | 2.6 | 1.7 |
| MDP ° C. | 58.7 | 39.0 | 48.2 | 57.7 | 39.8 | 33.1 |
| RXN comments | 180° C. to | 160° C. to | 200° C. to | 160° C. to | 160° C. to | No |
| | equib Vacuum | set AV then | set AV then | equib vacuum | equib vacuum | vacuum |
| | <30 torr for | vacuum of <30 | vacuum of <30 | <20 torr for | approx. 100 | |
| | entire | torr applied | torr applied | entire | torr for entire | |
| | reaction | until equib | until equib | reaction | reaction | |

*Amounts of HCO and Stearic acid are % by weight of the reaction mixture.

Examples 4A-F were prepared as represented in Table 4. They were run at different scales, for different times and utilized either a catalyst free or acid catalyst system as described in Examples 1 and 2. In addition, the degree and timing of the vacuum was varied. The reactions were typically allowed to continue until a stable state where equilibrium is reached or for an additional period of time. By varying the reactions conditions as shown the skilled artisan can manage the preference for the esterification of the hydroxy fatty acid vs transesterification of the triglyceride.

### Example 5

Examples 5 were prepared as represented in Table below. They show examples of inclusion of compositions of the present disclosure in lamellar and silicone free hair conditioners.

| | | | Control | | | | |
|---|---|---|---|---|---|---|---|
| Phase | Product | INCI Name | no PJ | 10A | 10B | 10C | 10D |
| A | DI water | DI Water | To 100 | To 100 | To 100 | To 100 | To 100 |
| | Glycerin | 1400 | 1400 | 600 | 1350 | 3525 | |
| | Actigum CS11 | 0.5 | 0.5 | 0.5 | 0.5 | 0.5 | 0.5 |
| | Star Design | Hydroxypropyl | 4.5 | 4.5 | 4.5 | 4.5 | 4.5 |
| | Care (Cargill) | Starch Phosphate | | | | | |
| | Star Design | Sodium | 2 | 2 | 2 | 2 | 2 |
| | Power (Cargill) | Octenylsuccinate | | | | | |
| | | and Hydroxypropyl | | | | | |
| | | Starch Phosphate | | | | | |
| | Example 3K | Natural PJ | 0 | 1.5 | 2 | 3 | 6 |
| C | Phenoxy | Phenoxy Ethanol | 1 | 1 | 1 | 1 | 1 |
| | Ethanol | | | | | | |
| | Vit E | Tocopherol | 0.1 | 0.1 | 0.1 | 0.1 | 0.1 |
| | | Acetate | | | | | |

| | | | | | |
|---|---|---|---|---|---|
| Stability/compatibility > 30 days | yes | yes | yes | yes | yes |
| Viscosity (cp) − Borrkfield TC-93 | 15150 | 4750 | 7400 | 9350 | 11900 |
| 20 rpm 1 min | | | | | |

The ingredients of each phase are mixed separately and added phases are added sequentially together with high shear mixing. The skilled artisan will easily be able to use known techniques and equipment to prepare this formulation. They will also be able to tailor the individual ingredients within the range (or add additional ingredients) to obtain their particularly desired texture.

### Example 6

Example 6 is a formulation of a powder blush cosmetic.

| | INCI Name | Wt Percent % |
|---|---|---|
| Oils | Sunflower, canola, coconut, hydrogenated | 40-50 |
| | canola, caprylic/capric triglyceride | |
| Waxes | Sunflower, candelilla, carnauba | 5-10 |
| Natural PJ | From Example 3K | 15-25 |
| Butters | Shea cocoa | 2-10 |
| Starches | Aluminum octenylsuccinate Zea Mays | 25-35 |
| | (corn) | |
| | Starch | |
| Pigments | Red 7, Yellow 5, TiO2 | 1-5 |
| Anti Oxidant | Vit E acetate | 0.1-1 |
| Preservative | Phenoxylethanol and Caprylyl Glycol | 0.1-1 |

### Example 7

Example 7 is a formulation of a cream or lotion.

| | | |
|---|---|---|
| Function | Raw Material | Wt Percent % |

Phase A

| | | |
|---|---|---|
| Water Phase | Water | QS to 100 |
| Rheology modifier | Actigum VSX20 Cargill | 0.3 |
| hydrocolloid | | |

Phase B

| | | |
|---|---|---|
| Oil phase | Clear Valley High Oleic | 20-40 |
| | Sunflower oil | |
| Emulsifier | Emulsun or lecithin | 3-8 |
| Skin Protectant | From Example 3K | 5 |
| Antioxidant | Vitamin E acetate | 0.2 |

Phase C

| | | |
|---|---|---|
| Preservative | PEHG | 1 |

The ingredients of each phase are mixed separately, and the phases are added sequentially together with high shear mixing. The skilled artisan will easily be able to use known techniques and equipment to prepare this formulation. They will also be able to tailor the individual ingredients within the range (or add additional ingredients) to obtain their particularly desired texture.

### Example 8

Example 8 is a formulation of bodywash or shampoo.

| | | |
|---|---|---|
| Function | Raw Material | Wt Percent % |

Phase A

| | | |
|---|---|---|
| Water Phase | Water | 45-55 |
| Humectant | Glycerine | 1-4 |
| Thickening Foam Boost | C*EmTEx 12688 | 1-10 |
| | (Cargill) | |

Phase B

| | | |
|---|---|---|
| Surfactant | Mackam 35-HA | 12-18 |
| Surfactant | Pureact SLG | 12-18 |
| Surfactant | Pureact SLI | 2-8 |

Phase C

| | | |
|---|---|---|
| Skin Protectant | From Example 3K | 1-10 |

Phase D

| | | |
|---|---|---|
| Preservative | Glycacil 2000 | 0.02-0.08 |
| Preservative | Phenoxyethanol | 0.1-2 |

The ingredients of each phase are mixed separately and added sequentially together with high shear mixing. The skilled artisan will easily be able to use known techniques and equipment to prepare this formulation. They will also be able to tailor the individual ingredients within the range (or add additional ingredients) to obtain their particularly desired texture.

### Example 9

Example 9 is a formulation of a lip balm.

| | INCI Name | Wt Percent % |
|---|---|---|
| Oils | Sunflower, canola, coconut, hydrogenated | 45-55 |
| | canola, caprylic/capric triglyceride | |
| Waxes | Sunflower, candelilla, carnauba | 10-20 |
| Natural PJ | From Example 3K | 15-25 |
| Butters | Shea cocoa | 15-25 |
| Anti Oxidant | Vit E acetate | 0.1-1 |
| Preservative | Phenoxylethanol and Caprylyl Glycol | 0.1-1 |

Manufacturing Process-Add all ingredients and start mixing and heating to 85° C. Make sure all waxes are melted and everything is uniform. Once everything is uniform and all ingredients have been added, make sure the temperature of the Lip Balm is between 80-85° C. Once it is between 80-85° C., pour the Lip Balm into the component relatively quickly to avoid settling and creating layers. Make sure to pour to the very top to avoid settling and concaving.

1. A personal care composition comprising a triglyceride component wherein: the triglyceride component comprises a mixture of triglycerides and wherein the mixture of triglycerides comprises individual triglycerides comprising one or more ester containing fatty acids and wherein the esters of the ester containing fatty acids are C8-C22 branched or straight chain fatty acid esters and wherein the triglyceride component has a drop melting point measured by AOCS Standard Procedure Cc 18-80 of between 30° and 70° C.

2. The personal care composition of claim 1 comprising between 50% and 100% weight percent of the triglyceride component.

3. The personal care composition of claim 1 comprising between 1% and 50% weight percent of the triglyceride component.

4. The personal care composition of claim 1 wherein the natural oil-glyceride component has an acid value between 0.5 and 20.

5. The personal care composition of claim 1 wherein the triglyceride component has a hydroxyl value between 10 and 90.

6. The personal care composition of claim 1 wherein the C8-C22 branched or straight chain fatty acid comprises stearic acid, palmitic acid, or mixtures thereof.

7. The personal care composition of claim 6 wherein the C8-C22 branched or straight chain fatty acid esters consist essentially of stearic acid and palmitic acid.

8. A personal care composition comprising a triglyceride component wherein: the triglyceride component comprises a mixture of triglycerides and wherein the mixture of triglycerides comprises individual triglycerides comprising one or more ester containing fatty acids and wherein the esters of the ester containing fatty acids are C8-C22 branched or straight chain fatty acid esters and wherein the composition contains between 0.5% and 10% combined monoglycerides and diglycerides.

9. The personal care composition of claim 8 comprising between 50% and 100% weight percent of the triglyceride component.

10. The personal care composition of claim 8 comprising between 1% and 50% weight percent of the triglyceride component.

11. The personal care composition of claim 8 wherein the triglyceride component has an acid value between 0.5 and 20.

12. The personal care composition of claim 8 wherein the triglyceride component has a hydroxyl value between 10 and 90.

13. The personal care composition of claim 8 wherein the C8-C22 branched or straight chain fatty acid comprises stearic acid, palmitic acid, or mixtures thereof.

14. (canceled)

15. The personal care product of claim 1, which is a body wash, face wash, shampoo, conditioner, combing cream, skin moisturizer, skin lotion, lip moisturizer, or cosmetic.

16. The personal care composition of claim 1, wherein the triglyceride component is the esterification product of: 55 wt % to 85 wt % of a fully hydrogenated castor oil, 15 wt % to 45 wt % a C8-C22 branched or straight chain fatty acid.

17. The personal care composition of claim 1, wherein the triglyceride component is the esterification product of: 55 wt % to about 85 wt % of a fully hydrogenated castor oil, 15 wt % to 45 wt % a C8-C22 branched or straight chain fatty acid and 5 wt % to 15 wt % a hydrogenated natural oil other than castor oil.

18. The personal care composition of claim 17, wherein the hydrogenated natural oil is hydrogenated soybean oil.

19. The personal care composition of claim 1, wherein the triglyceride component exhibits one or more rheological properties selected from a melt drop point of 35° C. to 70° C., a cone penetration at 25° C. of greater than 20 or from 20 to 100 or from 60 to 90 Dmm ( 1/10 of mm), kinetic viscosity at 100° C. of 5 mm²/s to 35 mm²/s, a congealing point of 25° C. to 45° C., or combinations thereof.

20. The personal care composition of claim 1, the composition include one or more of the following: (i) an acid value of less than 20.0; (ii) between 2% to 7 wt %; of combined monoglycerides and diglycerides, or (iii) an iodine value of less than 10.0.
